(19) United States
(12) Patent Application Publication
(10) Pub. No.: US 2026/0088182 A1
(43) Pub. Date: Mar. 26, 2026

(54) PREDICTION METHOD AND PREDICTION APPARATUS FOR LUNG FUNCTION CURVE AND STORAGE MEDIUM

(71) Applicant: XEEK (Xiamen) Medical Equipment Co., Ltd., Xiamen (CN)

(72) Inventors: Jingping ZHENG, Xiamen (CN); Mingjun HE, Xiamen (CN); Qingsheng WU, Xiamen (CN); Yishi LI, Xiamen (CN); Haimin LIU, Xiamen (CN); Zhufeng WANG, Xiamen (CN)

(21) Appl. No.: 19/217,339

(22) Filed: May 23, 2025

(30) Foreign Application Priority Data
Mar. 27, 2024 (CN) 202410361345.3

(51) Int. Cl. G16H 50/30

(52) U.S. Cl. CPC G16H 50/30

(57) ABSTRACT

The present disclosure provides a prediction method and prediction apparatus for a lung function curve and a storage medium. The prediction method includes: obtaining a flow rate at M sampling points; and inputting the flow rate at the M sampling points into a prediction model; and the construction of the prediction model includes: constructing a Taylor series expansion of an N-variable K^{th}-order equation, and representing, through the Taylor series expansion, a relationship between a flow rate at a t^{th} sampling point of a lung function curve and a flow rate at N sampling points preceding the t^{th} sampling point. In the above technical solution, a finite number of valid data points can be used to predict true lung function curve data.

### CROSS-REFERENCE TO RELATED APPLICATIONS

This application claims priority to Chinese Patent Application 202410361345.3, filed on Mar. 27, 2024, which is incorporated herein by reference.

### TECHNICAL FIELD

The present disclosure relates to prediction for lung function curves, and in particular, to a prediction method and prediction apparatus for a lung function curve and a storage medium.

### BACKGROUND

The pulmonary function test is an extremely important test in the diagnosis of respiratory system diseases. The pulmonary function test has an extremely important significance for the evaluation and treatment, and is the first and most critical evaluation index. However, the pulmonary function test is conducted in a high level of cooperation with the patient, otherwise it is difficult to obtain the valid data for the pulmonary function test, and the elderly or poorly educated patients are misdiagnosed easily due to poor quality control of test data. At present, there is a demand for prediction of complete lung function curves using a small amount of sample point data, but there is no existing program that can achieve a similar prediction function.

### SUMMARY

Embodiments of the present disclosure provide a prediction method and prediction apparatus for a lung function curve and a storage medium, to predict the true lung function curve data of a to-be-tested subject only based on a finite number of valid data points in the front portion of a pulmonary function test.

In order to achieve the above objective, according to an aspect, a prediction method for a lung function curve is provided. The prediction method includes:
obtaining a flow rate at M sampling points collected during expiration of a to-be-tested subject in a pulmonary function test; and
inputting the flow rate at the M sampling points into a pre-constructed and trained prediction model for a lung function curve, and predicting a lung function curve of the to-be-tested subject via the trained prediction model for a lung function curve,
where the construction of the prediction model for a lung function curve includes the following steps:

constructing a Taylor series expansion of an N-variable K^{th}-order equation, and representing, through the Taylor series expansion, a relationship between a flow rate at a t^{th} sampling point of the lung function curve during the expiration in the pulmonary function test and a flow rate at N sampling points preceding the t^{th} sampling point, where N, K, and t are natural numbers greater than or equal to 1, K≤N, and M≥N; and
the training of the prediction model for a lung function curve includes the following steps:
constructing a training dataset of the prediction model for a lung function curve by using data of a plurality of lung function curves that satisfy a predetermined qualification condition for quality control; and
training the prediction model for a lung function curve through the training dataset and a predetermined neural network algorithm until a predetermined training target is satisfied.

Preferably, in the prediction method, the relationship between the flow rate at the t^{th} sampling point of the lung function curve during the expiration in the pulmonary function test and the flow rate at the N sampling points preceding the t^{th} sampling point is expressed with the following Taylor series expansion of an N-variable K^{th}-order equation:

$$f ( t ) = {{a}_{{( t - 1 )}^{K}} [ f ( t - 1 ) ]}^{K} + {{a}_{{( t - 1 )}^{( K - 1 )}} [ f ( t - 1 ) ]}^{( K - 1 )} + … + {a}_{( t - 1 )} f ( t - 1 ) + {b}_{( t - 1 )} + … + {{a}_{{( t - N )}^{K}} [ f ( t - N ) ]}^{K} + {{a}_{{( t - N )}^{( K - 1 )}} [ f ( t - n ) ]}^{( K - 1 )} + … + {a}_{( t - N )} f ( t - N ) + {b}_{( t - N )}$$

f(t) represents the flow rate at the t^{th} sampling point; f(t−1) represents a flow rate at a (t−1)th sampling point; f(t−N) represents a flow rate at a (t−N)^{th} sampling point; a₍ₜ₋₁₎_{^{K}} to a₍ₜ₋₁₎ represent coefficients of terms of a corresponding order in f(t−1) respectively; a_{(t-N)}_{^{K}} to a_{(t-N)} represent coefficients of terms of a corresponding order in f(t−N) respectively; and b₍ₜ₋₁₎ to b_{(t-N)} represent corresponding constant terms respectively.

Preferably, in the prediction method, the data of the plurality of lung function curves that satisfy the predetermined qualification condition for quality control includes:
lung function curves that are of different genders, different ages, and/or different BMI ranges and that satisfy the predetermined qualification condition for quality control.

Preferably, in the prediction method, the predetermined neural network algorithm is a recurrent neural network algorithm.

Preferably, in the prediction method, the predetermined training target is that validation accuracy of prediction data for a same lung function curve is higher than a predetermined accuracy threshold.

Preferably, in the prediction method, the training the prediction model for a lung function curve through the training dataset and a predetermined neural network algorithm includes:
defining a starting equation for training and a termination equation for training, where the termination equation is an N-variable K^{th}-order equation; the starting equation is an N1-variable K1^{th}-order equation; and N1≤N, and K1≤K;
during training, when a number of variables and a number of orders of an equation do not reach a number of variables and a number of orders of the termination equation, determining whether a predicted value satisfies a predetermined validation criterion; if the predicted value does not satisfy the predetermined validation criterion, increasing the number of variables of the equation and/or the number of orders of the equation, and continuing the training; or if the predicted value satisfies the predetermined validation criterion, continuing to determine whether the predicted value satisfies the predetermined training target, terminating the training when the predicted value satisfies the predetermined training target, increasing the number of variables of the equation and/or the number of orders of the equation when the predicted value does not satisfy the predetermined training target, and continuing the training, where the validation criterion is that a difference between the predicted value and a true value is within a predetermined error threshold range; and
during the training, when both the number of variables and the number of orders of the equations reach the number of variables and the number of orders of the termination equation and the predicted value does not satisfy the predetermined validation criterion and/or the predetermined training target, adjusting a parameter of the predetermined neural network algorithm, adjusting the starting equation and/or the termination equation, and continuing the training.

Preferably, in the prediction method, the predetermined accuracy threshold is ≥95%; and the predetermined error threshold range is [−5%, +5%].

According to another aspect, a prediction apparatus for a lung function curve is provided. The prediction apparatus includes a memory and a processor, where the memory stores at least one program segment, and the at least one program segment is executed by the processor, to implement the prediction method in the foregoing description.

According to still another aspect, a computer-readable storage medium is provided, where the computer-readable storage medium stores at least one program segment, and the at least one program segment is executed by a processor, to implement the prediction method in the foregoing description.

The above technical solutions have technical effects:

In the technical solutions of embodiments of the present disclosure, by collecting a specific number of lung function curve data satisfying quality control, the prediction model for a lung function curve that is constructed through the Taylor series expansion in advance, that is, a relationship model between points in the lung function curve, is trained and tested through the predetermined neural network algorithm, to predict data points at a subsequent sampling moment based on a plurality of sampling data point at a preceding moment, thereby effectively predicting the lung function curve of the to-be-tested subject.

Further, an invalid lung function curve can be effectively predicted.

This resolves the misdiagnosis caused by inaccurate data collected when the to-be-tested subjects such as a patient are unable to properly perform an effective pulmonary function test. In addition, the pulmonary function monitoring process is simplified, which is conducive to the screening of pulmonary functions of the whole population.

### BRIEF DESCRIPTION OF DRAWINGS

FIG. 1 is a schematic diagram of a pulmonary function test in a conventional technology;

FIG. 2 is a schematic flowchart of a prediction method for a lung function curve according to an embodiment of the present disclosure;

FIG. 3 is a schematic flowchart of training a prediction model for a lung function curve through a prediction method for a lung function curve according to an embodiment of the present disclosure; and

FIG. 4 is a schematic diagram of a structure of a prediction apparatus for a lung function curve according to an embodiment of the present disclosure.

### DESCRIPTION OF EMBODIMENTS

To further illustrate the embodiments, the present invention provides accompanying drawings. The accompanying drawings, as a part of the present disclosure, are mainly used to illustrate the embodiments, and can explain the operating principles of the embodiments with reference to the related descriptions in this specification. With reference to such content, those of ordinary skill in the art can understand other possible implementations and the advantages of the present disclosure. Components in the drawings are not drawn to scale, and similar reference numerals are usually used to represent similar components.

The present disclosure will be further described below with reference to the accompanying drawing and specific implementations.

A basic measurement of the pulmonary function test is that the to-be-tested subject inhales maximally, then exhales with maximal effort until expiration is complete. FIG. 1 is a schematic diagram of a pulmonary function test in a conventional technology. In FIG. 1, PEF represents a highest expiratory flow; and FEF25%, FEF50%, and FEF75% represent instantaneous flows at 25%, 50%, and 75% of vital capacity during forced exhalation respectively. TLC represents a total lung capacity; and RV represents residual volume. Embodiments of the present disclosure focus on the prediction study of expiratory phase data during the pulmonary function test; and a pulmonary function conclusion is determined only based on the expiratory phase data.

During the expiration in the pulmonary function test, a working principle of a lung is as follows: The diaphragm relaxes and ascends, the abdominal muscles and internal intercostal muscles contract, enabling ribs to move downward. This compresses space in the lung, to expel air from the lung. After the air in the lung is expelled, movements are influenced only by the initial kinetic energy acquired during expulsion, the morphology of the respiratory tract, and the subsequent molecules of exhaled air.

The inventors of the present disclosure find that the expiration in a pulmonary function test can be understood by means of the showerhead principle. A head of a showerhead may use different forms as analogies to the respiratory tract morphology. When a water pressure and the showerhead are fixed, the water flow trajectory is fixed. When showerhead closing speeds are the same, the movement trajectory of the final water flow in the showerhead is fixed. That is, the magnitude of the subsequent water flow affects the trajectory of the water flow, such that the trajectory of the water flow can be predicted based on a water flow at the beginning.

In addition, a human respiratory tract is a relatively stable structure. Based on the above principle, the inventors of the present disclosure speculate that in the pulmonary function test, a flow rate at a moment after expiration can be predicted based on a flow rate at a moment before expiration. An N-variable K^{th}-order equation can express a relationship between a flow rate at a preceding moment and a flow rate at a subsequent moment. Because the water flow trajectory is fixed and variations of the water flow at different showerhead closing speeds are relatively continuous, it is speculated that this equation is a linear equation. Taylor series is a tool for solving an approximate value of a complex equation.

The inventors of the present disclosure construct, through the N-variable K^{th}-order equation and neural network algorithms such as a recurrent neural network algorithm (RNN), an automated model for predicting lung function curve data in the presence of a limited sample size. The inventors of the present disclosure speculate that the N-variable K^{th}-order equation is a linear equation. In some embodiments, the N-variable K^{th}-order equation is represented through a Taylor series expansion of the N-variable K^{th}-order equation.

A Taylor series expansion of a function f(x) is shown as follows:

$$f ( x ) = {\sum }_{n = 0}^{+ ∞} \frac{{f}^{( n )} ( 0 )}{n !} {x}^{n}$$

In embodiments of the present disclosure, a relationship between a flow rate at a preceding sampling point and a flow rate at a subsequent sampling point during expiration in the pulmonary function test is represented through the Taylor series expansion. In a specific implementation, a relationship between a flow rate at a t^{th} sampling point of the lung function curve during the expiration in the pulmonary function test and a flow rate at N sampling points preceding the t^{th} sampling point is represented through the Taylor series expansion, N, K, and t are natural numbers greater than or equal to 1, and K≤N. In a specific implementation, a relationship between a flow rate, that is, a flow rate f(t) during expiration, at a sampling time point t and a flow rate at N sampling points preceding t is represented through the following Taylor series expansion:

$$( t ) = {{a}_{{( t - 1 )}^{K}} [ f ( t - 1 ) ]}^{K} + {{a}_{{( t - 1 )}^{( K - 1 )}} [ f ( t - 1 ) ]}^{( K - 1 )} + … + {a}_{( t - 1 )} f ( t - 1 ) + {b}_{( t - 1 )} + … + {{a}_{{( t - N )}^{K}} [ f ( t - N ) ]}^{K} + {{a}_{{( t - N )}^{( K - 1 )}} [ f ( t - n ) ]}^{( K - 1 )} + … + {a}_{( t - N )} f ( t - N ) + {b}_{( t - N )}$$

f(t) represents the flow rate at the t^{th} sampling point such as the sampling time point t; f(t−1) represents a flow rate at a (t−1)th sampling point; f(t−N) represents a flow rate at a (t−N)^{th} sampling point; a₍ₜ₋₎_{^{K}} to a₍ₜ₋₁₎ represent coefficients of terms of a corresponding order in f(t−1) respectively; a_{(t-N)}_{^{K}} to a_{(t-N)} represent coefficients of terms of a corresponding order in f(t−N) respectively; and b₍ₜ₋₁₎ to b_{(t-N)} represent corresponding constant terms respectively.

The construction of a Taylor series expansion of a two-variable quadratic equation, that is, a quadratic equation in two variables, is illustrated by using an example of N=2, K=2:

$$F ( x , y ) = ax * x + b x + c + d * y * y + e * y + f ,$$

where a, b, and c in the equation represent a quadratic coefficient, a linear coefficient, and a constant term coefficient in a variable x respectively; and d, e, and f in the equation represent a quadratic coefficient, a linear coefficient, and a constant term coefficient in a variable y respectively.

Sampling point data for lung function training are [f1, f2, f3, f4 . . . fn], where fn represents a sampling value at an n^{th} flow rate. x,y is replaced with f(t−1),f(t−2) to construct an equation that predicts the flow rate f(t) at the t^{th} sampling point:

$$f ( t ) = {k}_{11} ( t - 1 ) * f ( t - 1 ) * f ( t - 1 ) + {k}_{12} ( t - 1 ) * f ( t - 1 ) + b ( t - 1 ) + {k}_{21} ( t - 2 ) * f ( t - 2 ) * f ( t - 2 ) + {k}_{22} ( t - 2 ) * f ( t - 2 ) + b ( t - 2 ) ,$$

where f(t) represents a value of flow rate at the t^{th} sampling point; k₁₁ represents a quadratic coefficient of f(t−1); k₁₂ represents a linear coefficient of f(t−1); k₂₁ represents a quadratic coefficient of f(t−2); k₂₂ represents a linear coefficient of f(t−2); b(t−1) represents a constant term coefficient of f(t−1); and b(t−2) represents a constant term coefficient of f(t−2).

### Embodiment 1

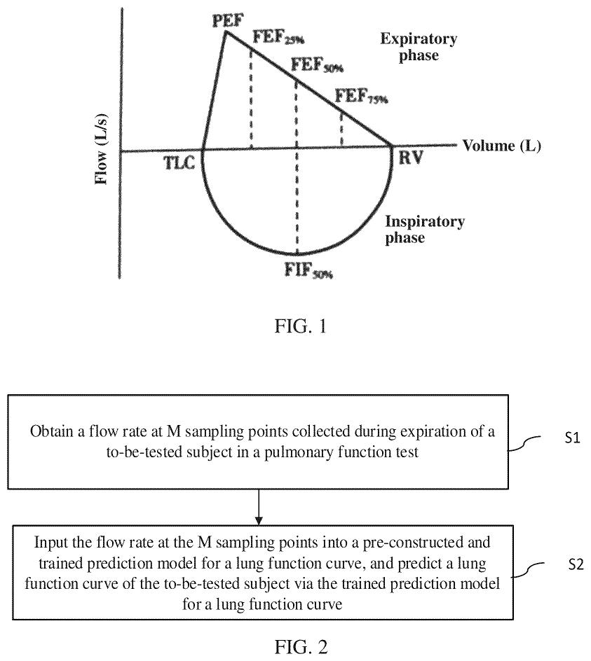

FIG. 2 is a schematic flowchart of a prediction method for a lung function curve according to an embodiment of the present disclosure. As shown in FIG. 2, the prediction method in this embodiment includes the following steps.
S1: Obtain a flow rate at M sampling points collected during expiration of a to-be-tested subject in a pulmonary function test, where M is a natural number greater than or equal to 1.
S2: Input the flow rate at the M sampling points into a pre-constructed and trained prediction model for a lung function curve, and predict a lung function curve of the to-be-tested subject via the trained prediction model for a lung function curve.

The construction of the prediction model for a lung function curve includes the following steps:

constructing a Taylor series expansion of an N-variable K^{th}-order equation, and representing, through the Taylor series expansion, a relationship between a flow rate at a t^{th} sampling point of the lung function curve during the expiration in the pulmonary function test and a flow rate at N sampling points preceding the t^{th} sampling point, where N, K, and t are natural numbers greater than or equal to 1, K≤N, and M≥N.

The training of the prediction model for a lung function curve includes the following steps:
constructing a training dataset of the prediction model for a lung function curve by using data of a plurality of lung function curves that satisfy a predetermined qualification condition for quality control, where the data of the plurality of lung function curves that satisfy the predetermined qualification condition for quality control includes: lung function curves of different genders, different ages, and/or different BMI ranges that satisfy the predetermined qualification condition for quality control; and
training the prediction model for a lung function curve through the training dataset and a predetermined neural network algorithm until a predetermined training target is satisfied, where the predetermined neural network algorithm is a recurrent neural network (RNN) algorithm.

In a specific implementation, the predetermined training target is that validation accuracy of prediction data for a same lung function curve is higher than a predetermined accuracy threshold. Preferably, the accuracy threshold is ≥95%. In a preferable implementation, the accuracy threshold is equal to 95%.

### Embodiment 2

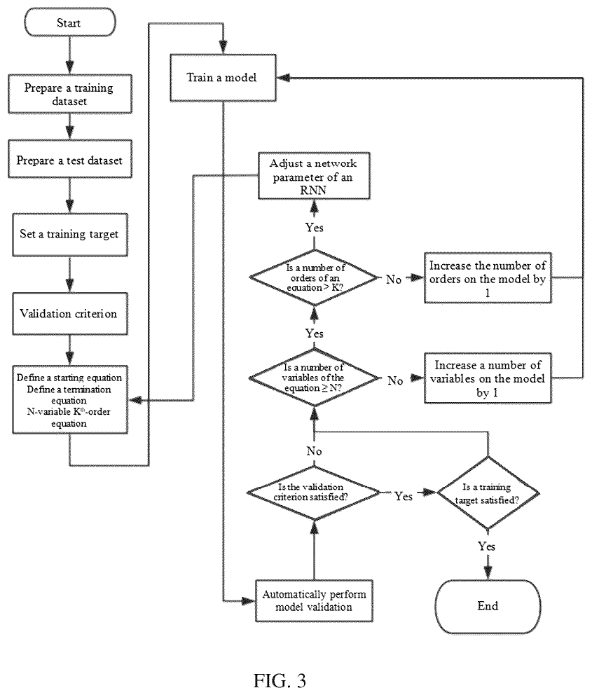

FIG. 3 is a schematic flowchart of training, through a training dataset and an RNN algorithm, a prediction model for a lung function curve in a prediction method for a lung function curve according to an embodiment of the present disclosure, that is, training an N-variable K^{th}-order equation that is used to represent a relationship between a preceding data point and a subsequent data point in a lung function curve and that is represented through a Taylor series expansion.

As shown in FIG. 3, the training of the prediction model for a lung function curve in this embodiment includes the following steps:
preparing a training dataset, where in a specific implementation, the training dataset includes lung function curve data that are of different genders, different ages, and/or different BMI ranges and that satisfy the predetermined qualification condition for quality control;
preparing a test dataset, where the test dataset is the same as the training dataset, except that the test dataset does not participate in the model training but is used for model validation;
setting a training target, where the training target is that validation accuracy of prediction data of a same curve is greater than 95%;
setting a validation criterion, where in a specific implementation, because an allowable error range of a flow rate in a lung function guideline is ±5%, the validation criterion defines that a difference between a predicted value and a true value cannot exceed ±5%; and if the difference between the predicted value and the true value is less than +5%, the prediction is accurate; and
defining a starting equation for training and a termination equation for training, where the starting equation is an N1-variable K1^{th}-order (that is, K1^{th} degree) equation, the termination equation is an N-variable K^{th}-order equation, N1≤N, and K1≤K.

In a specific implementation, the starting equation is defined as a five-variable linear equation. To be specific, a flow rate at a subsequent sampling point, that is, a 6^{th} sampling point, is predicted based on the preceding five sampling points. This is a preferred embodiment. However, in other implementations, the starting equation defining another number of variables and another number of orders can be used. For example, the starting equation is defined as a four-variable linear equation, a two-variable quadratic equation, or a five-variable quadratic equation, and details are not discussed herein.

In a specific implementation, the arithmetic power and the timeliness of prediction are considered. For example, in order to enable a data sampling frequency not to exceed 1 second, it may set that the number of orders of the termination equation does not exceed five orders, that is, the maximum degree of any variable in the Taylor series equation does not exceed the fifth order. This is a preferred embodiment. However, in other implementations, the termination equation defining another number of variables and another number of orders can be used. For example, the termination equation is a five-variable quartic equation, and details are not described herein.

The training and validating a training result, that is, a predicted value, including:
determining whether the predicted value satisfies the validation criterion; if the predicted value satisfies the validation criterion, further determining whether the training target is satisfied; if the training target is satisfied, ending the training; if the training target is not satisfied, determine whether a current number of variables of the equation is greater than or equal to N; if the current number of variables of the equation is not greater than or equal to N, increasing the number of variables on the model by 1, and continuing the training; if the current number of variables of the equation is greater than or equal to N, continuing to determine whether a current number of orders of the equation is greater than or equal to K; if the current number of orders of the equation is not greater than or equal to K, increasing the number of orders on the model by 1, and continuing the training; if the current number of orders of the equation is greater than or equal to K, adjusting a network parameter of an RNN, to redefine the starting equation for training and/or the termination equation for training; continuing the training until the number of variables and the number of orders of the equation are both greater than the number of variables and the number of orders of the termination equation; if the predicted value obtained through training satisfies both the predetermined validation criterion and the training target, ending the training; and obtaining a trained prediction model for a lung function curve.

The trained prediction model for a lung function curve can then be used to predict the lung function curve of the to-be-tested subject. In a specific implementation, subsequent lung function curve data can be predicted based on only a plurality of preceding valid data points of the to-be-tested subject obtained at the beginning of the pulmonary function test, that is, valid flow rate sampling points. The plurality of required valid data points corresponds to the number of variables of the termination equation on the trained prediction model for a lung function curve, that is, a number of previous valid data points for prediction needs to be greater than or equal to the number of variables of the termination equation on the prediction model.

In the prediction method for a lung function curve in embodiments of the present disclosure, the flow rate at the subsequent lung function data points can be predicted based on the plurality of previous valid flow rate data points, and the accuracy is high.

In embodiments of the present disclosure, further, validity of the invalid lung function curve can be predicted. In a specific implementation, a plurality of preceding data points in a lung function curve of which validity is to be determined may be taken as an input, and input to the trained prediction model, to obtain a predicted lung function curve. The lung function curve obtained through prediction is compared with the lung function curve of which validity is to be determined. If a deviation is within a predetermined deviation range, it may be determined that the lung function curve of which validity is to be determined is a valid lung function curve. If a deviation is not within a predetermined deviation range, it may be determined that the lung function curve of which validity is to be determined is an invalid lung function curve.

### Embodiment 3

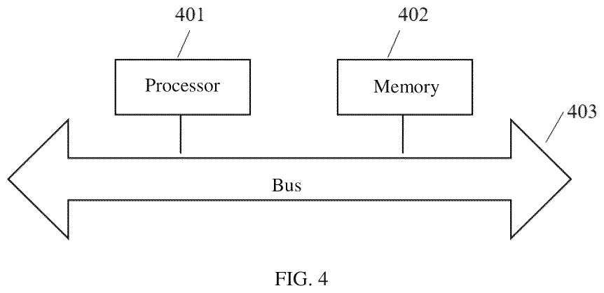

The present disclosure further provides a prediction apparatus for a lung function curve. As shown in FIG. 4, the prediction apparatus includes a processor 401, a memory 402, a bus 403, and a computer program stored on the memory 402 and executable on the processor 401. The processor 401 includes one or more processing cores. The memory 402 is connected to the processor 401 via the bus 403, and the memory 402 is used to store program instructions. The processor executes the computer program, to implement the steps in the foregoing method embodiment of the present disclosure.

Further, in an executable solution, the prediction apparatus for a lung function curve may be a computer unit, and the computer unit may be a computing device such as a desktop computer, a notebook, a palmtop computer, or a cloud server. The computer unit may include, but is not limited to, a processor and a memory. It is to be understood by those skilled in the art that a composition structure of the computer unit is merely an example of the computer unit, and does not limit the computer unit. The computer unit may include more or fewer components than those described above, some components may be combined, or different components may be used. For example, the computer unit may further include input and output units, a network access device, a bus, and the like. This is not limited in embodiments of the present disclosure.

Further, in an executable solution, the processor may be a central processing unit (Central Processing Unit, CPU), and may also be another general-purpose processor, a digital signal processor (Digital Signal Processor, DSP), an application specific integrated circuit (Application Specific Integrated Circuit, ASIC), a field-programmable gate array (Field-Programmable Gate Array, FPGA) or another programmable logic device, a discrete gate, a transistor logic device, a discrete hardware component, or the like. The general-purpose processor may be a microprocessor, or any conventional processor. The processor is a control center of the computer unit, and various parts of the whole computer unit are connected by using various interfaces and lines.

The memory may be configured to store the computer program and/or modules. The processor implements various functions of the computer unit by running or executing the computer program and/or modules stored in the memory and invoking data stored in the memory. The memory may mainly include a program storage area and a data storage area. The program storage area may store an operating system, an application program required by at least one function, and the like. The data storage area may store data created by a mobile phone, and the like. In addition, the memory may include a high-speed random access memory, and may further include a non-volatile memory, such as a hard disk, an internal storage, a plug-in hard disk, a smart media card (Smart Media Card, SMC), a secure digital (Secure Digital, SD) card, a flash card (Flash Card), at least one magnetic disk storage device, a flash memory device, or another volatile solid-state storage device.

### Embodiment 4

The present disclosure further provides a computer-readable storage medium. The computer-readable storage medium stores a computer program, and the computer program is executed by a processor to implement the steps of the method in the foregoing embodiments of the present disclosure.

The module/unit integrated in the computer unit, if implemented in the form of a software functional unit and sold or used as a stand-alone product, may be stored in a computer-readable storage medium. Based on such an understanding, all or some of processes for implementing the method in the foregoing embodiments can be completed by a computer program instructing related hardware. The computer program may be stored in a computer-readable storage medium. The computer program is executed by a processor to perform steps of the foregoing method embodiments. The computer program includes computer program code, and the computer program code may be in a form of source code, a form of object code, an executable file or some intermediate forms, and the like. The computer-readable medium may include: any physical entity or apparatus capable of carrying computer program code, a recording medium, a USB flash drive, a mobile hard disk drive, a magnetic disk, an optical disc, a computer memory, a read-only memory (ROM, Read-Only Memory), a random access memory (RAM, Random Access Memory), a software distribution medium, and the like. It should be noted that, the content included in the computer-readable medium may be added or deleted properly according to the legislation and the patent practice in the jurisdiction.

### Embodiment 5

The present disclosure further provides a computer program product. The computer program product stores a computer program, and the computer program is executed by a processor to implement the steps of the method in the foregoing description.

Although the present disclosure is specifically illustrated and described in combination with preferred implementation solutions, those skilled in the art should understand that various changes may be made to the present disclosure in terms of forms and details without departing from the spirit and scope of the present disclosure defined in the appended claims, which shall fall within the protection scope of the present disclosure.

1. A prediction method for a lung function curve, comprising:
obtaining a flow rate at M sampling points collected during expiration of a to-be-tested subject in a pulmonary function test; and
inputting the flow rate at the M sampling points into a pre-constructed and trained prediction model for a lung function curve, and predicting a lung function curve of the to-be-tested subject via the trained prediction model for a lung function curve,
wherein construction of a prediction model for a lung function curve comprises the following steps:
constructing a Taylor series expansion of an N-variable K^{th}-order equation, and representing, through the Taylor series expansion, a relationship between a flow rate at a t^{th} sampling point of the lung function curve during the expiration in the pulmonary function test and a flow rate at N sampling points preceding the t^{th} sampling point, wherein N, K, and t are natural numbers greater than or equal to 1, K≤N, and M≥N; and
training of the prediction model for a lung function curve to yield the trained prediction model for a lung function curve comprises the following steps:
constructing a training dataset of the prediction model for a lung function curve by using data of a plurality of lung function curves that satisfy a predetermined qualification condition for quality control; and
training the prediction model for a lung function curve through the training dataset and a predetermined neural network algorithm until a predetermined training target is satisfied.

2. The prediction method according to claim 1, wherein the relationship between the flow rate at the t^{th} sampling point of the lung function curve during the expiration in the pulmonary function test and the flow rate at the N sampling points preceding the t^{th} sampling point is expressed with the following Taylor series expansion of an N-variable K^{th}-order equation:
$$f ( t ) = {{a}_{{( t - 1 )}^{K}} [ f ( t - 1 ) ]}^{K} + {{a}_{{( t - 1 )}^{( K - 1 )}} [ f ( t - 1 ) ]}^{( K - 1 )} + … + {a}_{( t - 1 )} f ( t - 1 ) + {b}_{( t - 1 )} + … + {{a}_{{( t - N )}^{K}} [ f ( t - N ) ]}^{K} + {{a}_{{( t - N )}^{( K - 1 )}} [ f ( t - N ) ]}^{( K - 1 )} + … + {a}_{( t - N )} f ( t - N ) + {b}_{( t - N )}$$
f(t) represents the flow rate at the t^{th} sampling point; f(t−1) represents a flow rate at a (t−1)^{th} sampling point; f(t−N) represents a flow rate at a (t−N)^{th} sampling point; a(t−)K to a(t−) represent coefficients of terms of a corresponding order in f(t−1) respectively; a_{(t-N)}_{^{K}} to a_{(t-N)} represent coefficients of terms of a corresponding order in f(t−N) respectively; and b₍ₜ₋₁₎ to b_{(t-N)} represent corresponding constant terms respectively.

3. The prediction method according to claim 1, wherein the data of the plurality of lung function curves that satisfy the predetermined qualification condition for quality control comprises:
lung function curves that are of at least one of different genders, different ages, or different body mass index (BMI) ranges and that satisfy the predetermined qualification condition for quality control.

4. The prediction method according to claim 1, wherein the predetermined neural network algorithm is a recurrent neural network algorithm.

5. The prediction method according to claim 1, wherein the predetermined training target is that validation accuracy of prediction data for a same lung function curve is higher than a predetermined accuracy threshold.

6. The prediction method according to claim 5, wherein the training the prediction model for a lung function curve through the training dataset and a predetermined neural network algorithm comprises:
defining a starting equation for training and a termination equation for training, wherein the termination equation is an N-variable K^{th}-order equation; the starting equation is an N1-variable K1^{th}-order equation; and N1≤N, and K1≤K;
during training, when a number of variables and a number of orders of an equation do not reach a number of variables and a number of orders of the termination equation, determining whether a predicted value satisfies a predetermined validation criterion; when the predicted value does not satisfy the predetermined validation criterion, increasing at least one of the number of variables of the equation or the number of orders of the equation, and continuing the training; and when the predicted value satisfies the predetermined validation criterion, continuing to determine whether the predicted value satisfies the predetermined training target, terminating the training when the predicted value satisfies the predetermined training target, increasing at least one of the number of variables of the equation or the number of orders of the equation when the predicted value does not satisfy the predetermined training target, and continuing the training, wherein the predetermined validation criterion is that a difference between the predicted value and a true value is within a predetermined error threshold range; and
during the training, when both the number of variables and the number of orders of the equation reach the number of variables and the number of orders of the termination equation and the predicted value does not satisfy at least one of the predetermined validation criterion or the predetermined training target, adjusting a parameter of the predetermined neural network algorithm, adjusting at least one of the starting equation or the termination equation, and continuing the training.

7. The prediction method according to claim 6, wherein the predetermined accuracy threshold is ≥95%; and the predetermined error threshold range is [−5%, +5%].

8. A prediction apparatus for a lung function curve, comprising a memory and a processor, wherein the memory stores at least one program segment, and the at least one program segment is executed by the processor, to implement the prediction method according to claim 1.

9. A non-transitory computer-readable storage medium, wherein the non-transitory computer-readable storage medium stores at least one program segment, and the at least one program segment is executed by a processor, to implement the prediction method according to claim 1.
